(19) United States
(12) Patent Application Publication
(10) Pub. No.: US 2005/0065617 A1
(43) Pub. Date: Mar. 24, 2005

(54) System and method of performing ball and socket joint arthroscopy

(76) Inventors: Jose Luis Moctezuma de la Barrera, Freiburg (DE); Markus Jan Boehringer, Ehrenkirchen (DE); Adnan Cavus, Hoechenschwand (DE)

Correspondence Address: MCCRACKEN & FRANK LLP, 200 W. ADAMS STREET, SUITE 2150, CHICAGO, IL, 60606, US

(21) Appl. No.: 10/655,922

(22) Filed: Sep. 5, 2003

(51) Int. Cl. A61F 2/54

(52) U.S. Cl. 623/908; 606/102; 623/66.1

(57) ABSTRACT

A method of performing a total replacement surgery of a ball and socket joint of a patient using a surgical navigation system is performed by constructing intra-operatively a three dimensional model of the joint based on landmarks of the patient, by preparing the joint to receive implants, by placement of implants into the prepared joint and by determining range of motion and/or stability of the reconstructed joint. A system to perform a total replacement surgery of a ball and socket joint of a patient includes a surgical navigation system, a first circuit to construct intra-operatively a three dimensional model of the joint, a first tool to prepare the joint, a second tool to place an implant into the prepared joint, and a second circuit to determine range of motion and/or stability of the reconstructed joint. A virtual trialing or look ahead feature can also be included. A tool to locate the center of the canal of a limb includes an elongate body, a series of outwardly biased surfaces spaced around the elongate body and an interface to enable a tracking device to be attached to the body. A tool to guide the depth of the resection of a neck of a limb comprises a flat guide surface, a handle, and an interface to enable a tracking device to be attached to the tool.

### FIELD OF THE INVENTION

The present invention relates to a system and a method for performing a ball and socket joint arthroplasty or replacement. More particularly, this invention relates to a system and a method of performing ball and socket arthroplasty using an intraoperative construction of a model of the ball and socket joint.

### BACKGROUND OF THE INVENTION

There are two major types of ball and socket joints in human anatomy, two hip joints and two shoulder joints. There are a number of surgical approaches to repair of these ball and socket joints. For the hip joint, total hip arthroplasty (THA) or replacement surgery is used to provide increased mobility to patients who have significant problems with one or both of their hip joints, including injury, arthritis, bone degeneration, cartilage damage or loss, and the like. The classic THA surgery involves the dislocation of the hip joint following an incision to access the joint. Following dislocation of the joint, the femoral head is removed from the femur by cutting the femur through the femoral neck. The hip socket or acetabulum is then reamed out using a power tool and reaming attachment to remove the cartilage remaining within the acetabulum and to prepare the acetabulum to accept the acetabular implant component or cup. Typically, the reamer attachment is sized to prepare the acetabulum to accept a particular type of implant cup or component. The implant cup is held in place by cement, special screws and or by a mesh that accepts bone growth to firmly affix the cup to the pelvis.

The femur is then prepared by reaming the femoral canal using specialized rasps or similar instruments to shape the femoral canal to accept the fermoral stem implant. The femoral stem implant is then placed in the reamed out canal and affixed in place in a manner similar to the acetabular cup. The last step in the classic procedure is to attach a metal ball to the stem to act as the hip pivot point within the cup.

For the shoulder joint, replacement surgery is less common, and typical replacement surgery may only replace the ball of the humerus. In this case, the surgery typically will replace the ball of the humerus and sometimes make various levels of modification to the surface of the glenoid or socket.

Because the relative size and configuration of the implants can affect the length and offset of the leg or arm, care must be taken in the choice of the particular implants chosen. Often, prior to affixing the permanent implants in place, trial implants are placed in position to assist the surgeon to gauge the impact of the replacement surgery on the patient's mobility, range of motion, and quality of life. These issues include for the hip joint, making sure the leg length closely matches the length of the non-operative leg, making sure the offset of the replacement hip joint is satisfactory so that the appearance of the leg matches the non-operative leg, and making sure the replacement joint is sufficiently stable so that normal activity by the patient will not cause the hip to dislocate or cause the leg not to be able to properly support the patient during walking and other normal routine activities. For the shoulder, the length of the arm, the offset, and range of motion of the arm and shoulder must match the non-operative arm and shoulder and the operative shoulder must not dislocate under normal activity. One concern with the use of trial implants is that these trial devices are used after all preparation of the bone has taken place. If the trial indicates that the depth of the preparation is too great the surgeon is left with using implants of a different configuration to attempt to address the situation. This requires having a greater inventory of implants on hand before the surgery begins in order to address contingencies that may occur.

In addition, the classic surgical technique presents the surgeon with a number of other challenges. The use of surgical navigation and appropriate pre-surgical planning can minimize these challenges, but even with the use of these tools, care must be taken to insure appropriate modifications to the bone are made during the surgery. For instance with hip replacement surgery, it is necessary to prepare the acetabulum to a suitable depth to accept a certain acetabular implant cup, but at the same time avoid violating or compromising the medial wall of the acetabulum. At the same time, it is necessary to make sure that the acetabulum is prepared to properly accept the implant cup. If the cup does not sit well within the prepared acetabulum, for instance, if the prepared acetabulum is deeper than the depth of the cup or the cup can not be placed sufficiently deep within the acetabulum, the cup will either become loose over time or the pelvic structure may be damaged as the cup is impacted into place. There can be similar concerns for the shoulder if the glenoid is resurfaced or modified.

In addition to concerns relating to limb length and offset mentioned above, the surgeon currently must rely on mechanical guides to properly orient the implants in position relative to the patient's anatomy. Lastly, the surgeon must rely on their experience to assess the finished range of motion of the completed joint and the consequent potential for the joint to dislocate under normal everyday activities.

### SUMMARY OF THE INVENTION

One aspect of the present invention relates to a method of performing a total arthroplasty of a ball and socket joint of a patient using a surgical navigation system wherein the joint has a socket and a limb having a ball shaped head at a proximal end of the limb near the socket that includes constructing a three dimensional model of the joint intra-operatively using the surgical navigation system based on the patient's anatomical landmarks. The limb is prepared to receive a stem using the three dimensional model. Next, the stem is placed in the limb. Thereafter, the joint range of motion is determined.

A further aspect of the present invention relates to a method of performing a total arthroplasty of a ball and socket joint of a patient using a surgical navigation system wherein the joint has a socket and a limb having a ball at a proximal end of the limb near the socket that includes constructing a three dimensional model of the joint intra-operatively using the surgical navigation system based on the patient's anatomical landmarks. The limb is prepared to receive a stem using the three dimensional model. Next, the stem is placed in the limb. Thereafter, the stability of the joint is determined.

Another aspect of the present invention relates to a system for performing a total arthroplasty of a ball and socket joint on a patient that includes a surgical navigation system and a first circuit to construct a three dimensional model of the joint intra-operatively using the surgical navigation system based on the patient's anatomical landmarks. A first tool is used to prepare a limb to receive a stem, wherein the first tool can be tracked by the surgical navigation system to determine the position and orientation of the first tool and wherein the position and orientation of the first tool is tracked relative to the three dimensional model and a second tool is used to place the stem in the limb, wherein the second tool can be tracked by the surgical navigation system to determine the position and orientation of the second tool and wherein the position and orientation of the second tool is tracked relative to the three dimensional model. Lastly a second circuit determines the joint stability.

A still further aspect of the present invention relates a system for assisting in the performance of total arthroplasty of a ball and socket joint on a patient that includes a surgical navigation system, and a first circuit to construct a three dimensional model of the ball and socket joint intra-operatively using the surgical navigation system based on the patient's anatomical landmarks. The systems further includes a first tool to prepare a limb to receive a stem, wherein the first tool can be tracked by the surgical navigation system to determine the position and orientation of the first tool and wherein the position and orientation of the first tool is tracked relative to the three dimensional model and a second tool to place the stem in the limb, wherein the second tool can be tracked by the surgical navigation system to determine the position and orientation of the second tool and wherein the position and orientation of the second tool is tracked relative to the three dimensional model. In addition the system also includes a second circuit to determine a stability of the joint.

An additional aspect of the present invention relates to a method for performing a total arthroplasty of a ball and socket joint using a surgical navigation system. The method comprises the steps of constructing a three dimensional model of the joint and using the three dimensional model of the joint and data relating to implant components chosen from a database of hip joint implant components to provide a virtual trial of the joint. The method further includes the steps of preparing a limb to receive a stem using the three dimensional model. Lastly the method includes placing the stem within the prepared femur.

A further additional aspect of the present invention includes a system for performing a total arthroplasty of a ball and socket joint of a patient that includes a surgical navigation system and a first circuit to construct a three dimensional model of the joint. The system also includes a second circuit to provide a virtual trial of the joint using the three dimensional model of the joint and data relating to implant components chosen from a database of joint implant components and a first tool to prepare a limb to receive a stem, wherein the first tool can be tracked by the surgical navigation system to determine the position and orientation of the first tool and wherein the position and orientation of the first tool is tracked relative to the three dimensional model. Lastly, the system includes a second tool to place the stem in the limb, wherein the second tool can be tracked by the surgical navigation system to determine the position and orientation of the second tool and wherein the position and orientation of the second tool is tracked relative to the three dimensional model.

Another aspect of the present invention also includes a device to be used with a tracking device to locate the center of a canal of a limb that comprises an elongate body that can be inserted into the canal, a series of outwardly biased surfaces spaced around the elongate body and an interface attached to the body to enable a locating device capable of being tracked by a surgical navigation system to be affixed to the device.

Yet another aspect of the present invention includes a device to be used with a tracking device to locate a level of resection of a neck of a limb comprising a flat guide surface, a handle, and an interface attached to the handle to enable a locating device capable of being tracked by a surgical navigation system to be attached to the device

### BRIEF DESCRIPTION OF THE DRAWINGS

FIG. 1 is a schematic view of a surgical navigation system useful in the method of the present invention;

FIG. 2 is a flow diagram of a system to accomplish one embodiment of the method and system of the present invention;

FIG. 3 is a flow diagram of a further system to accomplish an additional embodiment of the present invention;

FIG. 4 is an anatomical view of the pelvis, hip joint, femur and tibia;

FIG. 5 is a frontal anatomical view of the pelvis, the hip joints and the tops of the femurs;

FIG. 6 is a flow diagram of one embodiment of the present invention for total hip replacement surgery;

FIG. 7 is a flow diagram of an embodiment of the virtual trial of the present invention for total hip replacement surgery;

FIG. 8 is a schematic view of a verification step of the three dimensional model created by an embodiment of the present invention;

FIG. 9 is an anatomic view of the femur showing the proximal shaft axis and the anatomical femoral axis;

FIG. 10 is a perspective view showing one embodiment of a device to locate the center of a canal of a limb of the present invention;

FIG. 11 is schematic view showing a resection guide of the present invention;

FIG. 12 is a schematic view showing a femoral impactor placed within the incision;

FIG. 13 is a diagrammatic view of a display screen showing aspects of the method and system of the present invention;

FIG. 14 is a diagrammatic view of a display screen showing further aspects of the method and system of the present invention;

FIG. 15 is a diagrammatic view of a display screen showing additional aspects of the method and system of the present invention;

FIG. 16 is a diagrammatic view of a display screen showing still further aspects of the method and system of the present invention;

FIG. 17 is a diagrammatic view of a display screen showing other aspects of the method and system of the present invention;

FIG. 18 is a diagrammatic view of a display screen showing still other aspects of the method and system of the present invention;

FIG. 19 is an anatomical view of a shoulder showing the location of various landmarks;

FIG. 20 is a flow diagram of an embodiment of the present invention for shoulder replacement surgery; and

FIG. 21 is a flow diagram of an embodiment of the present invention showing the use of a virtual trial for shoulder replacement surgery.

### DETAILED DESCRIPTION OF THE DRAWINGS

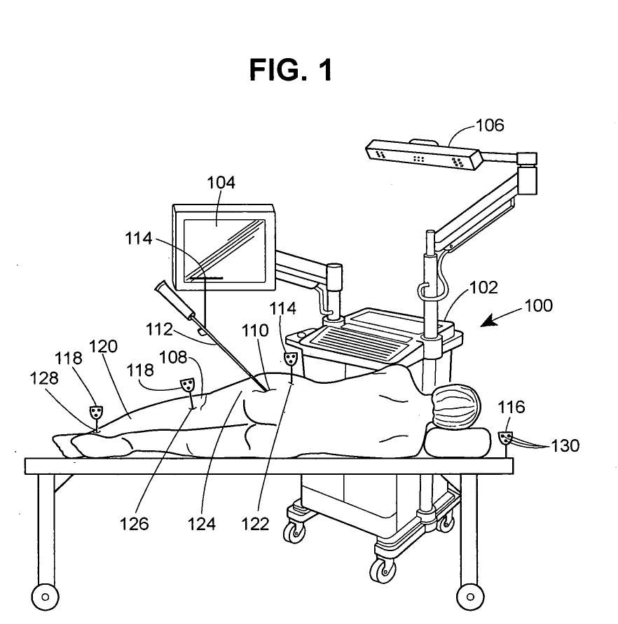

Referring to FIG. 1, a surgical navigation system 100 includes a personal computer 102 having a CPU (not shown), internal memory (not shown), and storage capacity (not shown), a monitor 104, and a camera array 106. The elements of the surgical navigation system 100 are well known to those of skill in the art and there are many commercially available systems that can be used in the method of the present invention, such as the surgical navigation system as disclosed in published U.S. patent application 2001/0034530, the disclosure of which is incorporated by reference. A patient 108 is prepared for the hip replacement surgery by a surgeon (not shown) making an incision 110. An instrument 112, such as an acetabular cup impactor, can be placed through the incision 110. A tracking device 114 that can be tracked by the camera array 106 is attached to the instrument 112.

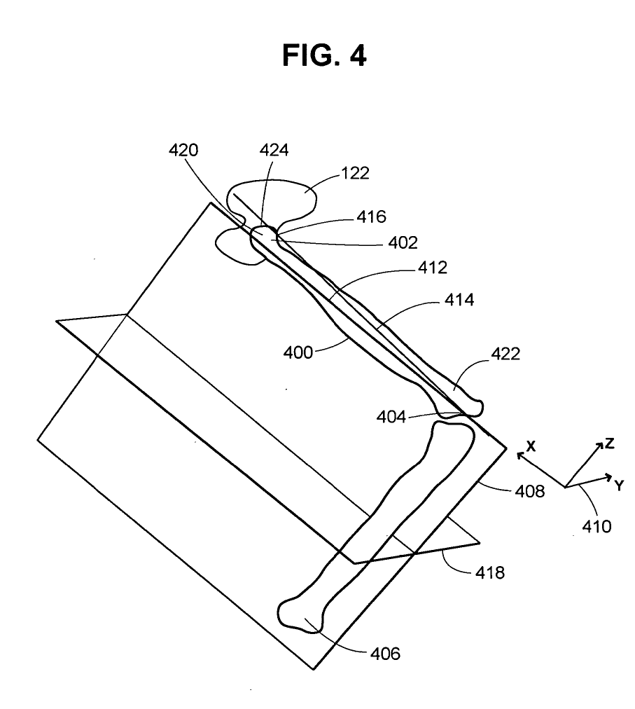

A reference tracking device 116 is preferably placed within the working volume of the camera array 106. In one embodiment of the present invention, tracking devices 118 are also invasively attached to the patient 108 at some or all of the following locations: a pelvis 122, an upper part 124 of a femur 400 (FIG. 4) or a lower part 126 of the femur 400 just above the knee joint on a leg 120. An additional optional location for the tracking device 118 is at an ankle 128 of the leg 120. For a preferred surgical navigation system 100 that utilizes three CCD cameras that can detect infrared light, the tracking devices 114, 116 and 118 all include three or more LED's 130 that emit infrared light so that the camera array 106 can see the locations of the LED's 130 on each of the tracking devices 114, 116, and 118. Based on a determination of the position and orientation of each of the tracking devices 114, 116, and 118, then by known techniques, the surgical navigation system 100 can determine the position and orientation of the anatomical structure or instrument to which the respective tracking device is attached.

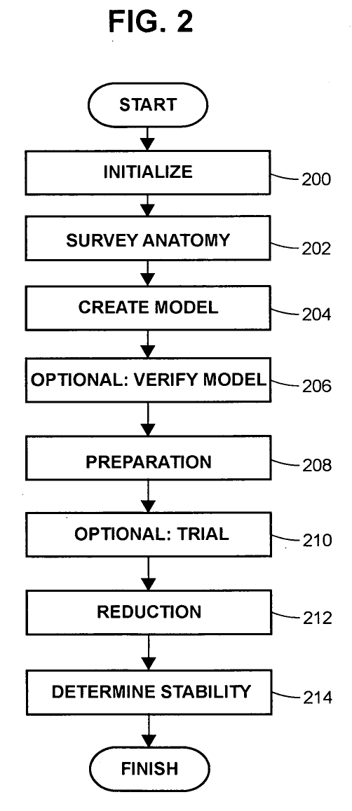

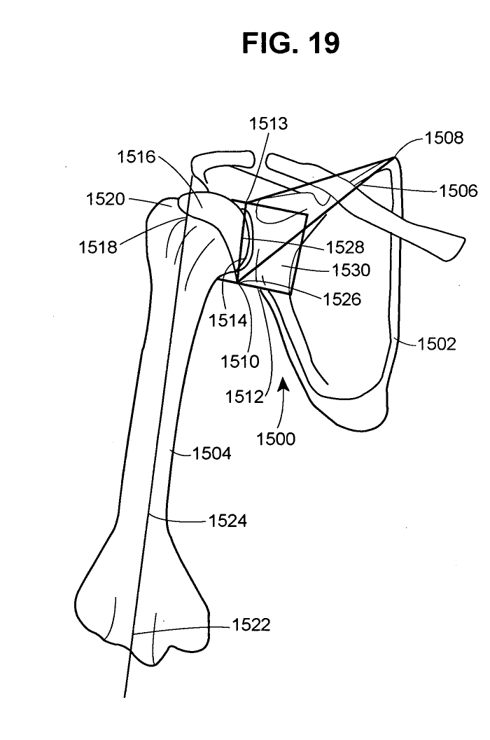

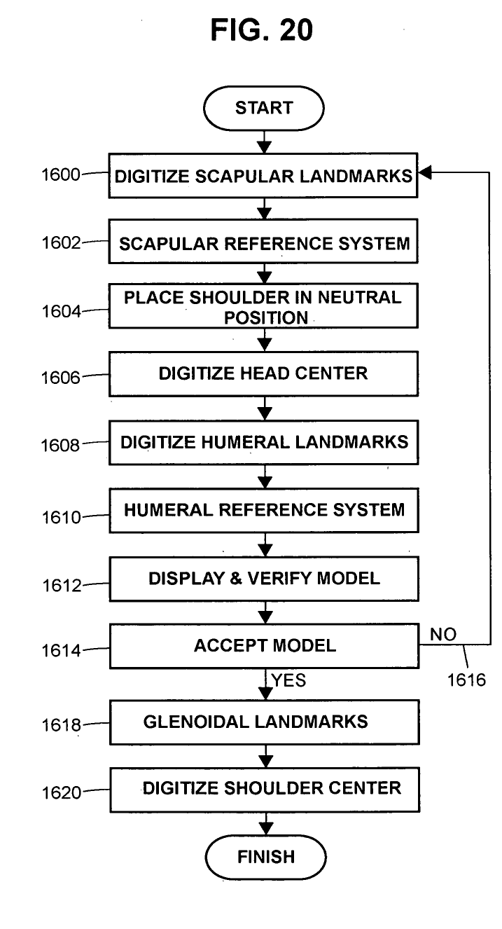

FIG. 2 shows a schematic diagram of one embodiment of the method and system of the present invention. After the system is initialized by a block 200 with the patient information, the pre-surgical planning information that includes information relating to the desired change of leg or arm length, if any, the desired change in joint offset, if any, and the like, the system proceeds to a block 202 that guides the surgeon though a survey of the patients anatomy so that the system can create a three dimensional model of the hip joint. The block 202 instructs the surgeon to either conduct a non-invasive survey of the joint or an invasive survey. Either type of survey is adequate for the preparation of a three dimensional model to further guide the surgeon through the choices to be made during the hip replacement or the shoulder replacement surgery. Details of the method of conducting the anatomy survey for hip surgery are discussed below with reference to FIGS. 4-6, and for shoulder surgery are discussed below relative to FIGS. 19-20.

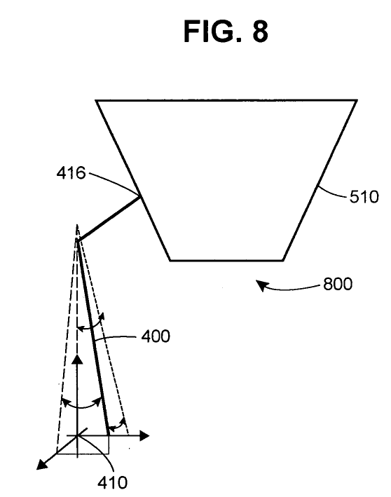

Based on the data determined by the block 202, a block 204 creates a three dimensional model e.g. for the hip comprising the acetabulum, the pelvis and the femur, including the femoral head. The details of the method of creating the three dimensional including the femoral head. The details of the method of creating the three dimensional model are discussed below relative to FIGS. 4-6. After the block 204 has created the three dimensional model, it is preferred that the method include a block 206 that verifies the model against the anatomy of the patient 108. The model is created using the biomechanical axes and the various planes of reference relative to the treated hip joint. A more detailed description of the verification step is discussed below with reference to FIG. 8.

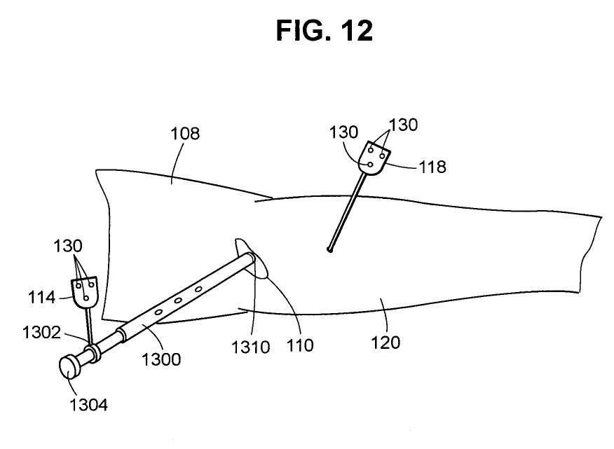

Once the model has been created by the block 204 and preferably verified by the block 206, the surgeon can proceed directly with elements of the hip replacement or the shoulder replacement surgery. In FIG. 2, a block 208 represents the preparation of the acetabulum and the femur for hip replacement surgery or the preparation of the humerus and possibly the glenoid for shoulder replacement surgery. The exact order of preparation is not important with regard to the method of the present invention. Either the femur or the acetabulum can be prepared first for hip surgery. Some surgeons have been trained to begin with the femur and some begin with the acetabulum. Also, the condition of the patient's hip joint may dictate the order of preparation. In a similar manner for shoulder surgery, the humerus is prepared and the glenoid may optionally be prepared. Again, the order of preparation is a matter of choice. After the joint has been prepared by the block 208, a block 210 represents the optional step of conducting a trial reduction of the joint. A trial reduction can provide the surgeon with an indication that the preparation will work well with the particular implant components chosen. If the trial reduction step is conducted, temporary implants having the same size as the final implants are placed into position in the prepared joint. The joint is temporarily repositioned and the surgeon manipulates the joint to determine the likely stability and other characteristics of the final implant. Either after the optional trial reduction of the block 210, or directly after the preparation of the joint by the block 208, a block 212 represents the final reduction of the joint by the surgeon. In the block 212, the surgeon will place the final implants into the prepared acetabulum and femur for hip replacement surgery or into the humerus and optionally the glenoid for shoulder surgery. The implants are secured in place using navigated techniques analogous to the preparation step. A more detailed discussion of the navigated final reduction is discussed below relative to FIGS. 1 and 12. After the trial or final reduction of the block 212 has been conducted, the surgeon will confirm the stability of the joint in a block 214. In the block 214, the surgeon will manipulate the joint to confirm that the joint will not dislocate under normal activities and that the range of motion is acceptable. Also the surgeon confirms the thereby attained leg length and offset. As part of this confirmation, the surgeon will look at the soft tissue surrounding the joint and also look at the potential for the reconstructed joint to dislocate.

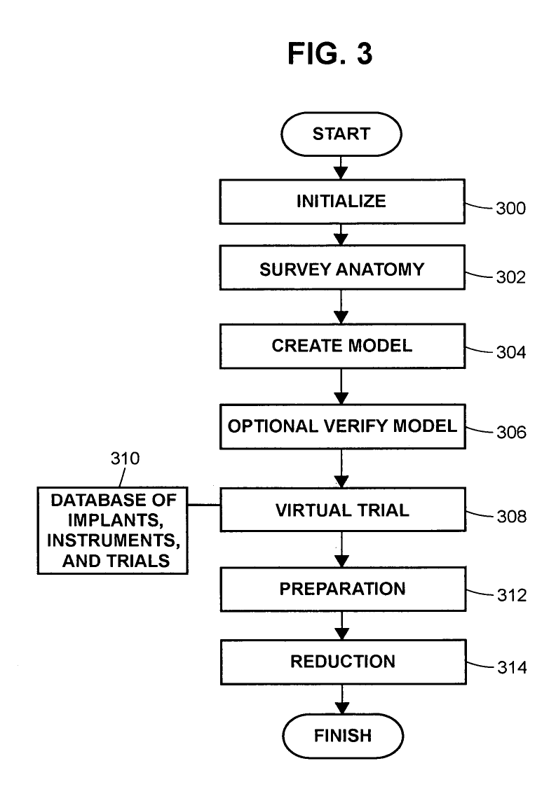

FIG. 3 shows a flow diagram of an alternative method and system of the present invention. In this alternative method and system, the blocks 300 to 306 are all performed in the same manner as blocks 200 to 206 and blocks 312 and 314 are conducted in the same manner as the blocks 208 and 212, respectively. At any time after the three dimensional model of the joint has been prepared by the block 304, the surgeon can do a virtual trial or look ahead as represented by a block 308 to predetermine the nature of the preparation necessary for the acetabulum or glenoid, the size and nature of the implant cup that will be used, the shape and depths of the broach of the femur or humerus for insertion of the stem, the length of the offset and the size of the ball to be used. Even though the block 308 is indicated at being done immediately after the block 306, it can be conducted alternatively, or in addition, after the preparation block 312 or the reduction block 314. The block 308 uses the data from the three dimensional model created by the block 304 and also uses data taken from a block 310 that includes a database containing data for a wide range of implant components, including the particular implant components that have been chosen by the surgeon, instruments and trial components. As noted previously, the database 310 contains values for the properties, dimensions and other parameters of many of the implant components typically used in total hip replacement and shoulder replacement surgery and the database 310 is stored within the computer 102. Alternatively, the block 308 can use a three dimensional model created, either in whole or in part, based on pre-surgical scan data.

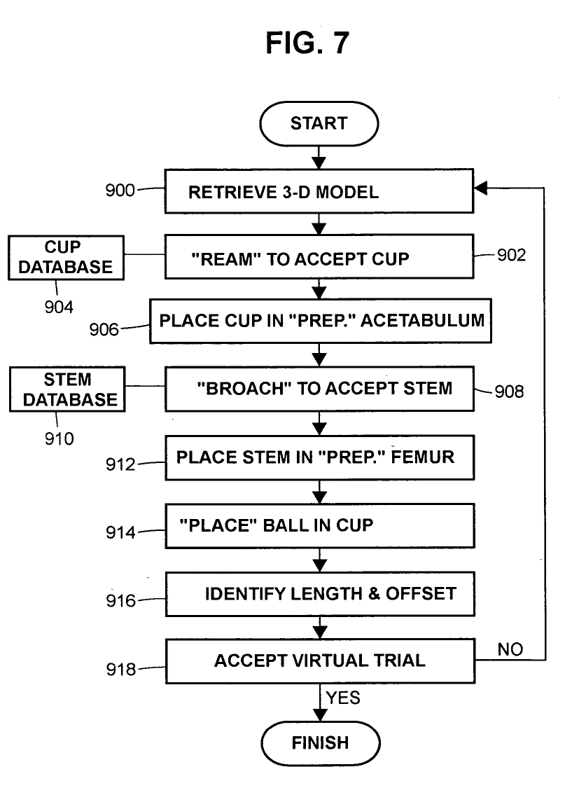

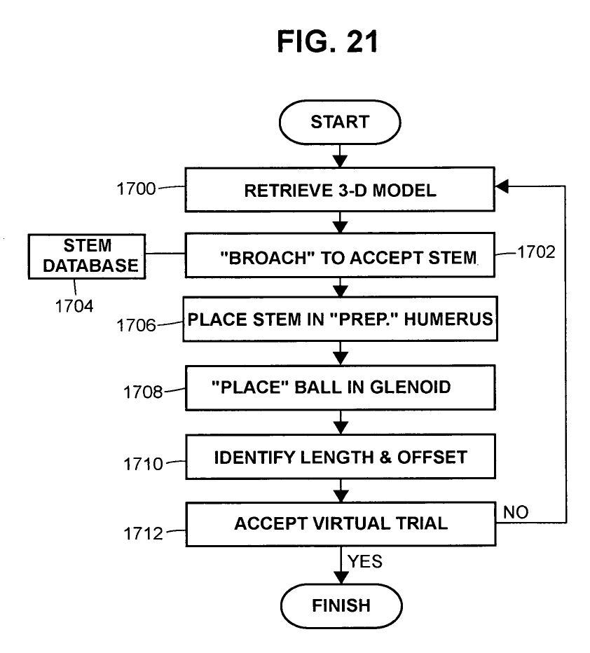

The look ahead or virtual trial of the block 308 allows the surgeon to assess offset, leg length, and the range of motion of the joint with the proposed implants in place before significant preparation of the bone has been done. The surgeon can simulate the preparation of the joint by indicating the nature and size of the reaming of the acetabulum and the broaching of the femur for hip replacement surgery and the humerus and optionally the glenoid for shoulder surgery. Based on the biomechanical axes and the various planes of reference, as well as the gaps in the structure, the virtual trial can also estimate the effect of the soft tissue on the joint stability. Alternatively the surgeon can compare actual preparation of these structures to determine the optimum implant components to achieve the desired post surgical result. Therefore, using this technique, the surgeon can monitor the progress as the joint is prepared and make the minimum preparation necessary to achieve a satisfactory result. This will help the surgeon minimize post surgical damage to the underlying boney structure by reaming too deeply within the acetabulum leaving too thin a structure to properly support the patient or creating too thin a wall in the femur that also could create problems post surgically. Similar problems can be avoided in shoulder replacement surgery. The look ahead or virtual trial of the block 308 can be done at any time during the surgical technique. It can be used as an alternative to an actual trial of the joint or in place of an actual trial of the implant components. A more detailed description of the look ahead or virtual trial is discussed below relative to FIG. 7 for hip replacement surgery and FIG. 21 for shoulder surgery.

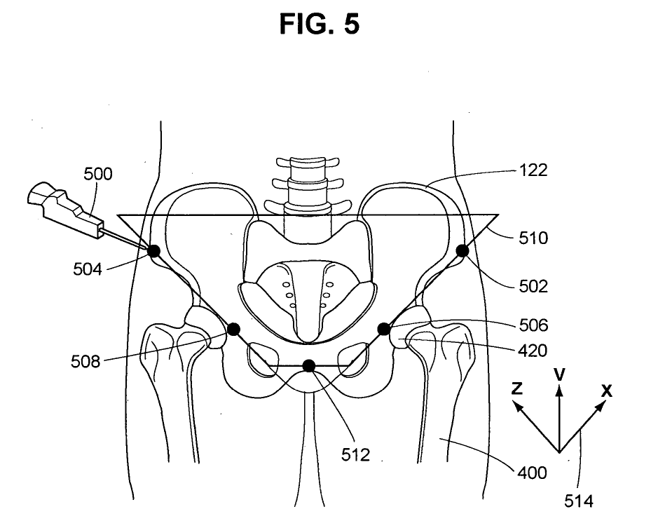

With reference to FIGS. 4 and 5, a non invasive survey will involve the surgeon using a properly calibrated trackable pointing device, as are well known to those skilled in the art of the use of surgical navigation systems. For instance, a suitable trackable pointer 500 is used, as disclosed in published U.S. patent application 2001/0034530, the disclosure of which is incorporated by reference. With reference to FIG. 5, the trackable pointer 500 is used to locate and digitize the left and right ASIS 502 and 504 and the left and right pubic tubercles 506 and 508. This is done by touching the point of the trackable pointer 500 to the anatomical landmark and notifying the surgical navigation system 100 to mark the location of the landmark. This is a well known technique done using surgical navigation systems. The identification of these anatomical landmarks allows the identification of a frontal plane 510. An alternate method of locating the frontal plane 510 is by locating and digitizing the suspensory ligament 512 and the left and right ASIS 502 and 504. A pelvic coordinate system 514 is created having an x-axis on the pelvic frontal plane 510 pointing from left to right, the y-axis normal to the pelvic frontal plane 510, and a z-axis perpendicular to the x and y axes. This later method of locating the frontal plane 510 is more robust if the patient 108 is obese. With reference to FIG. 4, the surgeon is prompted to place the hip joint in a neutral position. With a hip joint 416 in the neutral position, the location of the tracking devices 118 attached to the pelvis 122 and the femur 400 are determined and the transformation between the pelvic coordinate system 514 and a femoral coordinate system 410 is recorded by the surgical navigation system.

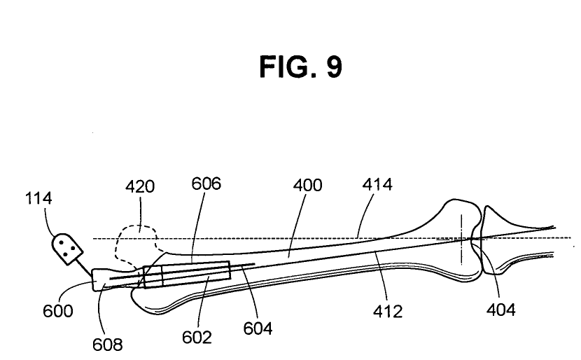

The piriformis fossa 402 of the treated femur 400 is digitized by touching the trackable pointer 500 to the piriformis fossa and notifying the surgical navigation system to record the location of the piriformis fossa 402. In a similar manner, the location of the popliteal fossa 404 is also digitized. After the popliteal fossa 404 has been digitized, the surgeon is instructed to flex the knee joint to 90 degrees. This will enable the calculation of a femoral sagittal plane 408 when the achilles midpoint 406 is digitized in the same manner as above. The femoral coordinate system 410 has the x-axis normal to the sagittal plane 408 pointing from left to right as shown, the z-axis is an anatomical axis 412 between the piriformis fossa 402 and the popliteal fossa 404, and the y-axis is perpendicular to the x-axis and the z-axis. FIG. 9 also shows the anatomical axis 412 in more detail and compares the anatomical axis 412 with a mechanical axis 414 of the leg 120.

To find a center of the hip joint 416, motion analysis is used. In this analysis, described in U.S. Pat. No. 5,611,353, the disclosure of which is hereby incorporated by reference, the tracking device 118 is attached to the leg 120 at a distal end 422 of the femur 400 and the femur 400 is rotated within view of the surgical navigation system 100. The system 100 tracks the tracking device 118 and digitizes a series of locations. From these locations, the center of the hip joint 416 can be located using the center of a sphere matched to the locations of the tracking device 118 recorded by the system 100. Any suitable sphere matching algorithm can be used to match the sphere, such as a least squares algorithm and the like. The tracking device 118 can be attached to the patient's leg using known technology. Even if the tracking device 118 is attached directly to the bone to perform this analysis, the procedure is still considered non-invasive since the incision to attach the tracking device 118 is quite small or it can be place within the incision without the necessity for a separate access through the skin to the bone. From the center of the hip joint 416, the frontal plane 510 and the anatomical axis 412 of the femur 400, the angulations and translations as e.g. offset and leg length changes of the hip joint can be determined.

As part of the surgical procedure, the digitization of the femur and the acetabulum can also be created invasively. After dislocation of the hip, the articular surface of the acetabulum can be digitized by taking the pointing device and tracing the surface of the articular surface of the acetabulum. Also, the shape of the fovea can also be digitized to enable the surgical navigation system to determine the proper depth of the preparation of the acetabulum.

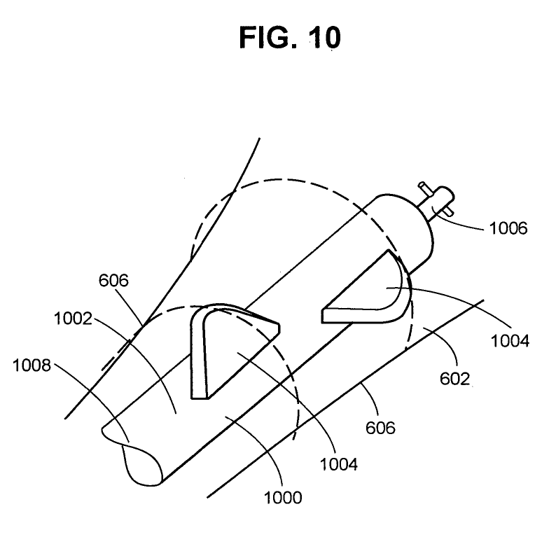

As shown in FIG. 9, the mechanical axis 414 of the femur 400 is the line between the center of a femoral head 420 and the popliteal fossa 404. The mechanical axis 414 is how the body bears the weight through the center of the hip joint 416 through the femur 400. After the femoral head 420 has been removed as part of the initial stage of the preparation of the femur 400, an instrument 600, such as a reamer, is placed within the proximal canal 602 and a proximal shaft axis 604 of the proximal canal 602 is digitized by referencing the inner walls 606 of the canal 602. This can be done using the instrument 600 that has a tracking device 114 attached. The instrument 600 must be small enough to fit within the canal 602 so that the axis 604 can be digitized. Alternatively an instrument 1000 as shown in FIG. 10 can be used to digitize the proximal canal 602. The instrument 1000 includes a body 1002 and a series of spring biased members 1004 spaced around the periphery of the body 1002. As the instrument 1000 is placed within the proximal canal 602 the spring biased members 1004 center the instrument 1000 within the canal 602. A tracking device similar to tracking device 114 is attached to the body 1002 by an interface lug 1006 attached to the distal end 1008 of the body 1002. The digitization of the proximal canal 602 provides the surgeon with a shift value 608 (FIG. 9) of the proximal shaft axis 604 of the proximal canal 602 from the anatomical axis 412 of the femur 400. From this shift value 608, the surgeon can determine the amount, if any, of varus/valgus relative to the proximal shaft axis 604. The amount of anteversion relative to the coronal femoral plane 416 can also be determined.

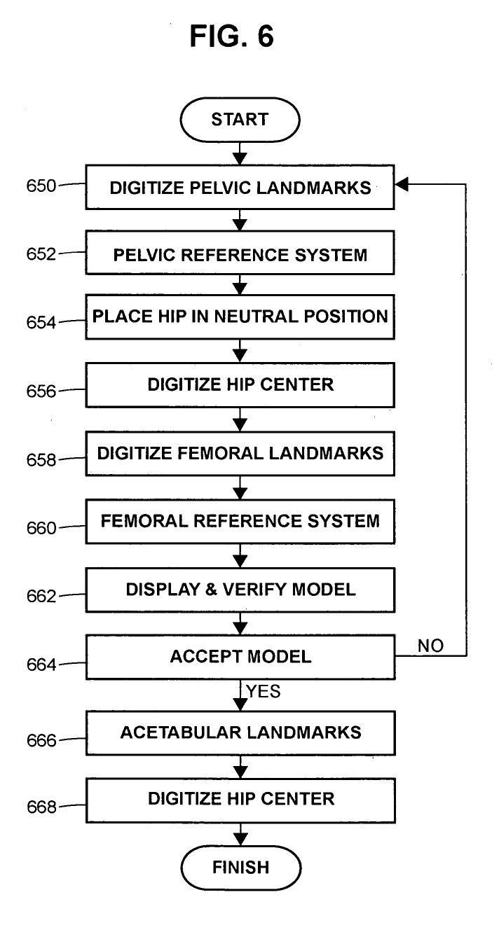

As shown in FIG. 6, a flow diagram of the method and system for conducting the anatomical survey and creation of the three dimensional model of the hip is shown. A block 650 prompts the surgeon to digitize a series of landmarks on the pelvis such as the left and right ASIS 502 and 504 and the left and right pubic tubercles 506 and 508, as discussed earlier. A block 652 then calculates the pelvic coordinate system with an x-axis pointing from the left ASIS to the right ASIS, the y-axis is normal to the pelvic frontal plane 510 and the z-axis is perpendicular to the x and y axes. Control then passes to a block 654 that instructs the surgeon to position the leg of the patient 108 in a neutral position as defined by the surgeon. Next, a block 656 determines the ipsilateral hip center in a manner as described above using rotation of the femur about the hip joint. After the center of the hip joint has been determined, control passes to a block 658 that instructs the surgeon to digitize the piriformis fossa 402, the popliteal fossa 404, and the achilles midpoint 406 as described above. Control then passes to a block 660 that calculates the sagittal plane and the femoral coordinate system. Thereafter, a block 662 displays the model on the display 104 and instructs the surgeon to manipulate the femur and compare the motion of the femur with the motion of the model on the display 104. If the motion of the femur matches the motion of the model, the surgeon will accept the model in a block 664. If the motion of the model does not match to the surgeon's satisfaction, the surgeon will not accept the model in the block 664 and the system will branch back to the block 650 to begin the routine again.

If the model is acceptable, control passes to a block 666 that instructs the surgeon to digitize the surface of the fovea and the articular surface of acetabulum after the hip joint has been dislocated. The digitization of the fovea enables the system to navigate the depth of the preparation of the acetabulum. The digitization of the articular surface of the acetabulum also provides an alternative method to determine the center of the hip joint. A block 668 enables the surgeon to choose the data for the center of the hip joint from the motion analysis data, the articular surface data or from the center of a navigated reamer that has been inserted into the acetabulum. At this point the survey and model creation module is completed.

A view of a model 800 of a hip joint 416 is shown in FIG. 8 that also shows how the hip joint 416 can be manipulated to verify the model that has been created. As shown in FIG. 8, the frontal plane 510 is tracked by the tracking device 118 that has been attached to the pelvis 122. The femur 400 is tracked by the tracking device 118 that has been attached to the lower femur 124. The display 104 shows a view of the model 800 and the surgeon will manipulate the patient's hip joint 416 and observe the motion of the model 800 on the display 104 relative to the femoral coordinate system 410. If the motion of the model 800 matches the motion of the actual joint, the model 800 will be accepted. It is also possible to quantitatively verify the model intra-operatively by digitizing actual landmarks such as the identification of the acetabular plane, and the like. The model can also be compared to pre-operative scans of various types, such as x-rays.

With reference to FIG. 7, the details of the method and system to conduct the virtual trial or look ahead are discussed. When the surgeon chooses to do a virtual trial or look ahead, a block 900 will retrieve the three dimensional model. If no model has been created or verified, the system will warn the surgeon that the look ahead is not available at that point. Control passes to a block 902 that imports the data from a block 904 of the implant database containing data on the chosen cup implant. The block 902 will perform a virtual preparation of the acetabulum to accommodate the implant cup chosen from the block 904. A block 906 will place the virtual cup within the virtual preparation of the acetabulum and provide the surgeon with data indicating the expected parameters of the resulting hip joint such as leg length and offset change, anteversion, and inclination. These values can be varied by the surgeon to simulate placing the cup within the prepared acetabulum in different attitudes. If these values are acceptable, control passes to a block 908 that imports the data from a block 910 of the implant database containing data on the chosen femoral stem implant and ball implant to match the cup implant. The block 908 then conducts a virtual preparation of the femur to accommodate the chosen stem implant. Control then passes to a block 912 that places the step in the virtually prepared femur. Next, if the surgeon accepts the virtual stem preparation, the system passes control to a block 914 that virtually places the implant ball within the implant cup and a display block 916 displays the expected values for the leg length change, varus and valgus and offset. Control then passes to a block 918 that requests acceptance of the look ahead or virtual trial. If the trial is acceptable, the routine exits. If the trial is not acceptable, control passes back to the block 900. Also, it is possible to interrupt the routine at any time during the routine to exit to the main program.

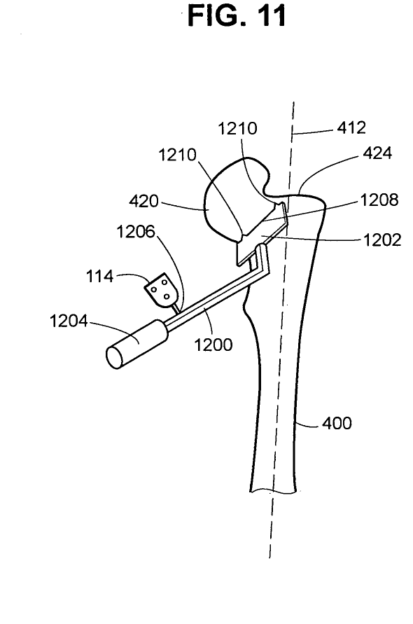

Another usage of the virtual trial is to aid the surgeon locating the correct angle and position for resection of the femoral neck. As shown in FIG. 11, the surgeon can use the surgical navigation system 100 and the three dimensional model that includes the location of the proximal axis and the dimensions and other characteristics of the proposed implant to correctly position a resection guide 1200. The resection guide 1200 has a guide surface 1202, a handle 1204, and an interface 1206 to enable a tracking device 114 to be attached to the resection guide 1200. The guide surface 1202 includes at a proximal end 1208 at least two teeth 1210 to enable the resection guide 1200 to be securely held in position. After the resection guide 1200 has been placed in the proper position, a surgical saw blade (not shown) is placed against the guide surface 1202. The resection guide 1200 can be placed in position based on the three dimensional model to provide the desired angle and height for the resection of the femoral neck 420 relative to the proximal shaft axis 604 and a top surface 424 of the femur 400. The height is computed as a function of the implant size, the center of the femoral head 420, proximal shaft axis 604 and offset and leg length changes that are required. The knowledge of the correct resection level is needed for surgical techniques where the access to the anatomy is limited when a minimally invasive single anterior incision approach is used for the hip replacement procedure.

After the acetabulum and the femur have been prepared as described above, the reduction step 212 is performed. In this step, the surgeon will permanently place the cup implant within the prepared acetabulum and the stem implant within the prepared femur. FIG. 1 shows the cup impactor 112 in position within the incision 110 at the hip of the patient 108. The proper cup implant has already been affixed to the distal end of the impactor 112 by conventional means, such as by a threaded interface. The tracking device 114 in cooperation with the surgical navigation system 100 provides the surgeon with information to enable the surgeon to position the implant cup properly within the acetabulum. The surgical navigation system 100 will indicate to the surgeon the distance the cup must still travel to be properly seated within the prepared acetabulum. The system will also indicate to the surgeon the orientation of the cup impactor 112 so that the abduction and version of the cup will provide maximum stability and range of motion to the patient. In a similar manner, as shown in FIG. 12 the stem 1310 is inserted in to the prepared femoral canal using a navigated stem impactor 1300. The stem impactor 1300 includes an interface 1302 to which the tracking device 114 can be attached. A head 1304 of the impactor 1300 is designed so that force can be applied to the impactor 1300 to properly place the stem 1310 into position within the proximal canal 602. In a manner similar to the cup impactor 112, the navigation system 100 will guide the surgeon so that the orientation of the stem 1310 will achieve the desired post surgical result. The advantage of navigating the insertion or impaction of the acetabular cup and/or the femoral stem is that the depth to sit or the depth of the implant within the prepared surface can be carefully controlled and monitored by the surgeon. In a similar manner, the orientation of the implant can also be controlled. This is especially useful where the implant is secured in position by an adhesive. In this situation, the adhesive can allow the implant to float within the prepared boney structure so that securing the implant in the correct orientation is much more difficult without the assistance and guidance of a surgical navigation system.

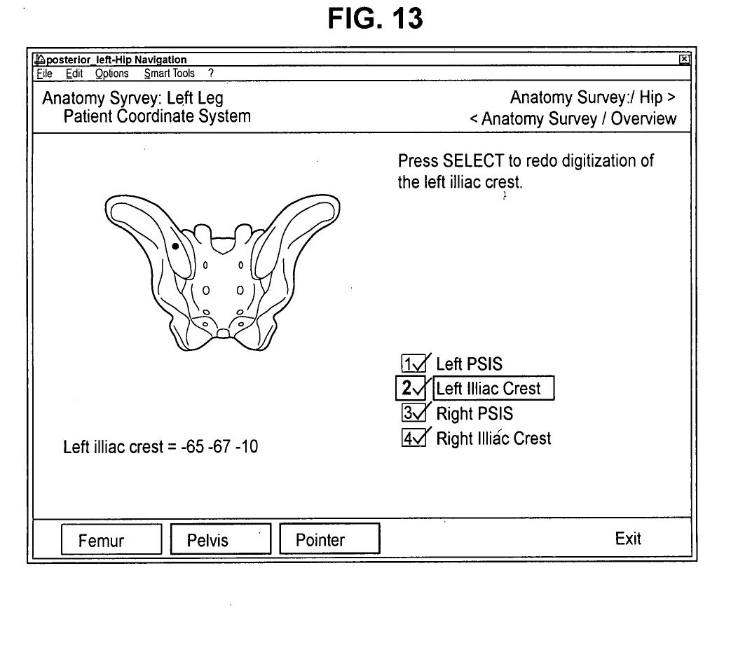

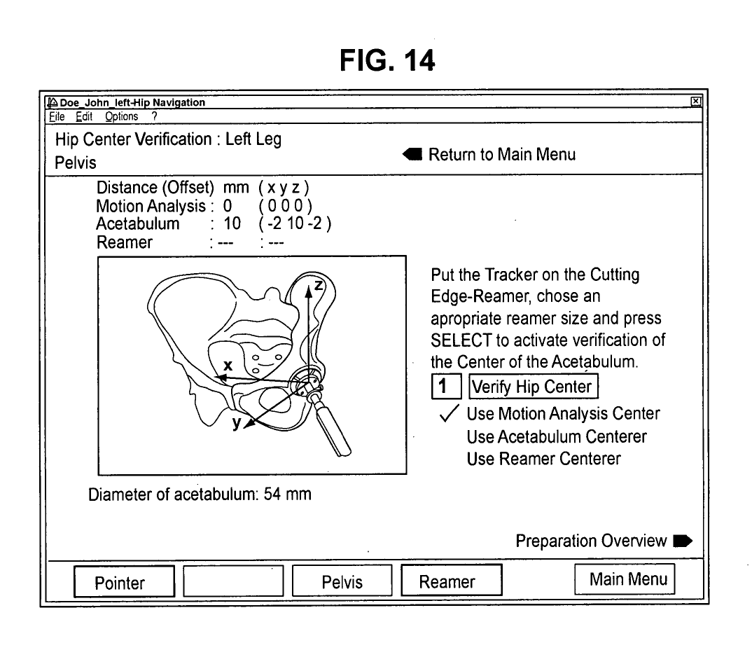

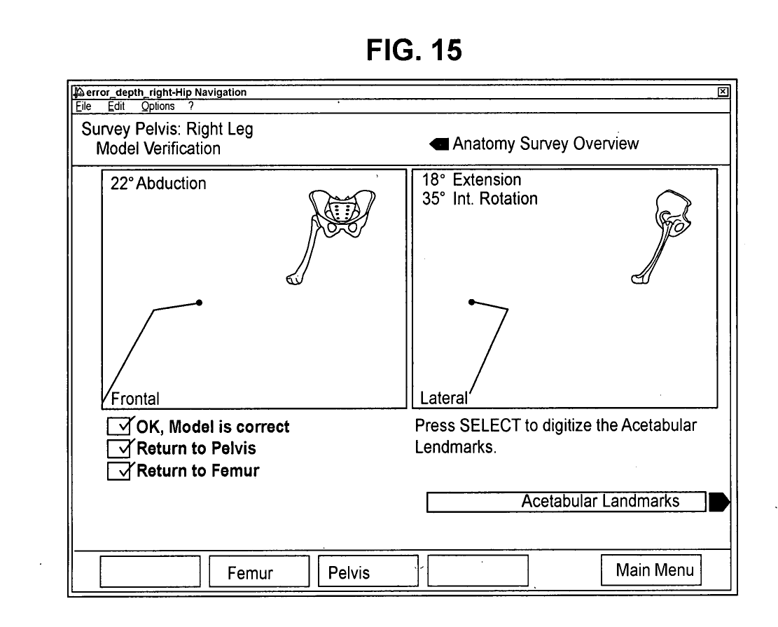

FIGS. 13-15 show a series of computer screen shots of a computer program that will enable the preparation of an intra-operative three dimensional model of the hip joint. As shown in FIG. 13, the system prompts the surgeon to digitize the left illiac crest as part of the creation of the three dimensional model. In FIG. 14 a screen is shown that assists the surgeon to locate the center of the acetabulum using an invasive model formation technique. In this instance, the reamer with an appropriate diameter is placed in the dislocated hip joint and the tracking device attached to the reamer is activated to locate the center of the sphere of the reamer cutting surface as described with reference to the block 666 of FIG. 6. FIG. 15 shows a screen to verify the model 800 as described with reference to the block 206 in FIG. 2, the block 662 in FIG. 6, and FIG. 8. The surgeon moves the leg 120 of the patient 108 and matches the motion of the leg 120 and the hip joint 416 to the motion of the model 800 on the display 104. Typically this verification is done before the hip is dislocated at the beginning of the surgical procedure.

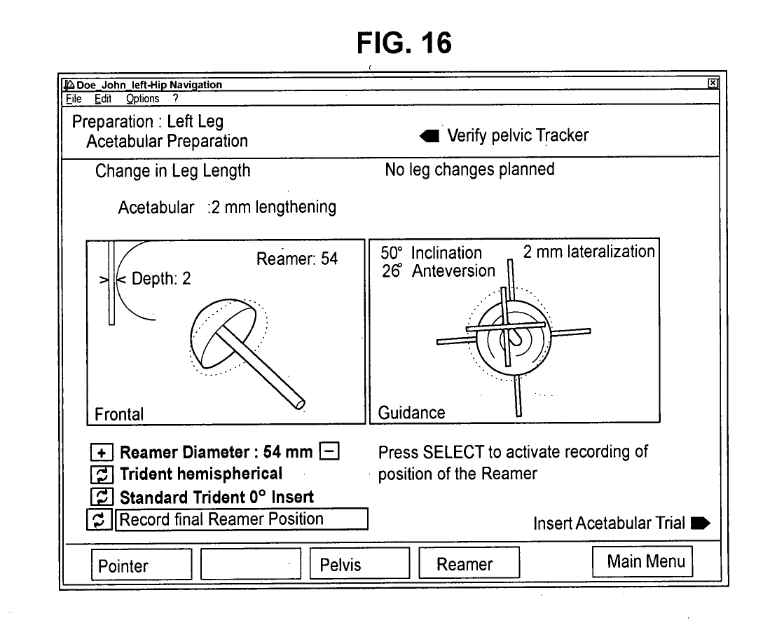

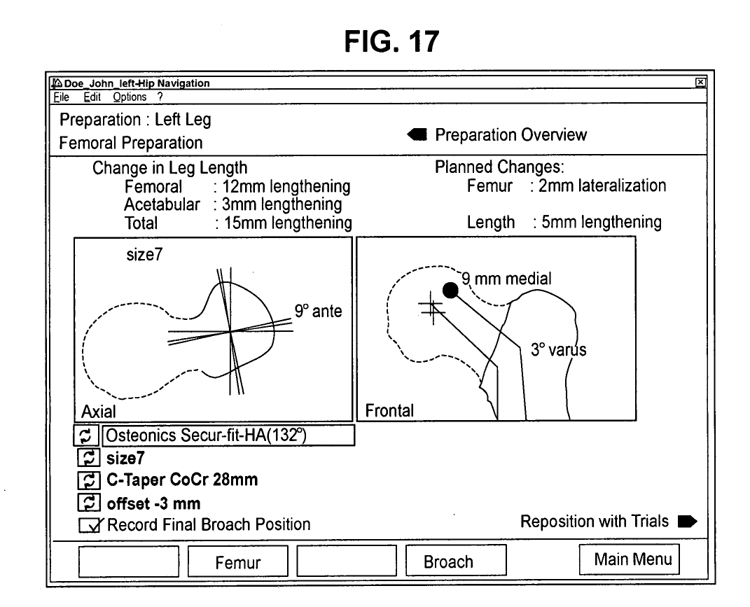

FIG. 16 shows a screen relating to the preparation of the acetabulum. The left pane of FIG. 16 shows that the reamer must still penetrate 2 mm into the structure of the acetabulum to achieve the desired post surgical result for the chosen acetabulum cup implant. The right pane of FIG. 16 shows the orientation of the reamer within the acetabular cup as determined by the surgical navigation system 100. FIG. 16 also shows the specifications of the reaming tool and the description of the proposed prosthesis cup. FIG. 16 also indicates that the surgeon can record the final position of the reamer within the prepared acetabulum when desired. Lastly, in the area above the left and right panes, the program shows that if the procedure were concluded at the present time, the leg would be lengthened by 2 mm versus a pre surgical plan of no lengthening of the leg. A similar screen will guide the surgeon to place the acetabular cup implant into the proper position as the cup is impacted into final position within the prepared acetabulum. FIG. 17 shows a screen that displays the potential change in hip and leg parameters based on a particular stem chosen from the database in comparison to the planned changes. FIG. 17 also shows both the axial and frontal views of the femoral preparation. A screen similar to FIG. 17 will also guide the surgeon in the proper depth and orientation of the stem implant as the stem is impacted into final position.

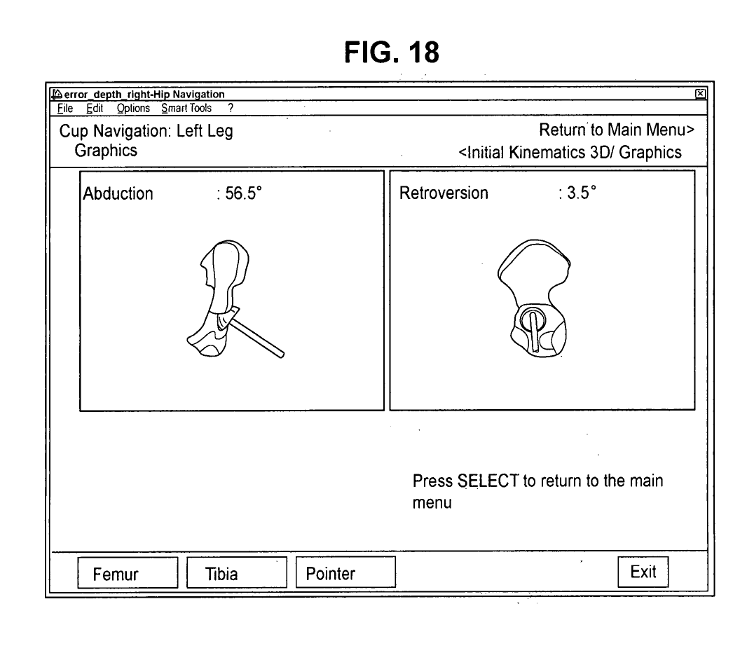

FIG. 18 shows a screen used by the surgeon to guide the placement of the acetabular cup within the prepared acetabulum. The left pane of FIG. 18 shows a view that indicates that in the current position, the cup will have an abduction of 56.5° and the right pane shows that the cup will have a retroversion of 3.5°.

FIG. 19 shows an anatomical view of a shoulder joint 1500 that is formed from a scapula 1502 and a humerus 1504 with the surrounding ligaments and muscle omitted for clarity. The landmarks used to create the three dimensional model for the shoulder joint 1500 are a coronal plane 1506 defined by a medial angle 1508 of the scapula 1502, a lateral angle 1510 of the scapula 1502, and a most superior aspect 1513 of a neck 1512 of the scapula 1502. The scapula includes a glenoid 1514 into which a head 1516 of the humerus 1504 fits. The humerus 1504 has an intertrochanteric groove 1518 that has a most superior aspect 1520. The humerus 1504 also has a midpoint 1522 between the coronoid fossa and the radial fossa. The midpoint 1522 and the most superior aspect 1520 of the intertrochanteric groove 1518 define an anatomical axis 1524 of the arm. Inclination of the glenoid 1514 is defined in reference to a line 1526 extending from the most superior aspect 1513 of the neck 1512 to a most inferior aspect 1528 of the neck 1512. Version of the glenoid 1514 is defined with reference to a plane 1530 perpendicular to the coronal plane 1506. If necessary, a sagittal plane can be defined by the anatomical axis 1524 and a midpoint of the volar radiocarpal ligament of the wrist (not shown).

The method and system for creating the three dimensional model for use in performing shoulder replacement surgery described in FIG. 20 is similar to that described with reference to FIG. 6. After initialization the system starts and proceeds to a block 1600 that instructs and guides the surgeon through the digitization of the scapular landmarks including the medial angle 1508, the lateral angle 1510, and the most superior aspect 1513 of the neck 1512. After these landmarks have been digitized, the system proceeds to a block 1602 that creates the scapular reference system including the coronal plane 1506, the line 1526 and the plane 1530. This reference system will also create a Cartesian coordinate system with the x-axis and the y-axis on the coronal plane 1506 and the z-axis perpendicular to the x and y axes. After the creation of the scapular reference system by the block 1602, the system proceeds to a block 1604 that instructs the surgeon to place the shoulder and arm in a neutral position. The system then continues to a block 1606 that guides the surgeon through the digitizing of the head 1516 of the humerus 1504. This is done in a manner similar to the digitization of the femoral head 420 in the hip joint 416 using suitable a sphere matching algorithm such as least squares and the like. The system then continues to a block 1608 that instructs and guides the surgeon through the digitization of the humeral landmarks, including the most superior aspect 1520 of the intertrochanteric groove 1518 and the midpoint 1522 between the coronoid fossa and the radial fossa. The system then proceeds to a block 1610 that creates the humeral reference system including the anatomical axis 1524 and the sagittal plane if needed.

At this point the system then proceed to a block 1612 that displays the model on the monitor 104 and instructs the surgeon to manipulate the arm to test the model and displays a block 1614 that allows the surgeon to accept or reject the model. If the motion of the model on the monitor 104 matches the motion of the arm and shoulder, the surgeon can accept the model in the block 1614 or if the motion does not match to the surgeon's satisfaction, the surgeon can reject the model in the block 1614. If the surgeon does not accept the model in the block 1614, the system will branch via a NO branch 1616 back to the block 1600 to begin the creation of the model. If the model is acceptable the system will proceed to a block 1618 that instructs the surgeon to digitize the glenoidal landmarks. Typically this is done in a manner similar to the digitization of the acetabulum described above after the shoulder has been dislocated. The system then proceeds to a block 1620 that instructs the digitization of the shoulder center of rotation in a manner similar to the digitization of the hip center of rotation described above. At this point the system then exits this routine and proceeds to the preparation of the shoulder to accept the implants. Alternatively, the surgeon can utilize the virtual trial as shown in FIG. 21 to test the proposed intervention before any significant changes are made to the patient's anatomy.

FIG. 21 describes the use of a virtual trial in the context of shoulder replacement surgery. At any time during the shoulder replacement surgery procedure, the surgeon can perform a virtual trial. This virtual trial can be performed before any significant modification is made to the patient's anatomy, after one of both of the humerus and glenoid has been prepared for acceptance of the implants, or after preparation of the humerus and/or glenoid but before the actual or trial implants are placed into the prepared structures. When the surgeon requests a virtual trial, the system will begin with a block 1700 that retrieves a previously prepared three dimensional model. The block 1700 will at this time check to determine the integrity of the three dimensional model and if the model is not considered acceptable by the system, the routine will exit and warn the surgeon that the virtual trial feature is not available. After the three dimensional model has been retrieved, the system will proceed to a block 1702 that will request the data on a suitable stem, either specified by the surgeon or suggested by the system based on the three dimensional model from a stem database 1704. The block 1702 will take the data relating to the stem chosen from the stem database 1704 and perform a virtual preparation or broaching of the humerus to accept the chosen stem. At this time, the system will go to a block 1706 that then virtually places the chosen stem into the virtually prepared humerus. Next, the system will in a block 1708 place a suitable ball onto the stem based on the available balls from the stem database 1704 and fit the stem and ball combination into the glenoid structure that has been prepared virtually. In shoulder replacement surgery, the glenoid preparation is often much more minimal than that done for hip surgery and a separate cup implant is often not used for shoulder replacement surgery. The stem and ball combination must be able to work with the minimally modified cartilage or tissue that remains in the glenoid after preparation. After placement, the system then come to a block 1710 that will determine the length and offset of the arm using the selected stem, ball and preparation. This is reported to the surgeon by the block 1710 and the surgeon is given an opportunity by a block 1712 to either accept or reject the virtual trial. If the trial is rejected the system will branch back to the block 1700 to begin the virtual trial again. If the trial is acceptable, the system will exit to the point in the procedure where the surgeon was when the virtual trial was started. Also, at any time during the entire virtual trial procedure, the surgeon can end the virtual trial and resume the procedure where the surgeon left off.

### Industrial Applicability

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications, which come within the scope of the appended claims, are reserved.

1. A method of performing a total arthroplasty of a ball and socket joint of a patient using a surgical navigation system wherein the joint has a socket and a limb having a ball shaped head at a proximal end of the limb near the socket comprising the steps of:
constructing a three dimensional model of the joint intra-operatively using the surgical navigation system based on the patient's anatomical landmarks;
preparing the limb to receive a stem using the three dimensional model;
placing the stem in the limb; and
determining the joint range of motion of the joint.

2. The method of claim 1 wherein the ball and socket joint is a hip and wherein the method includes the additional steps of:
preparing the socket to receive a cup using the three dimensional model; and
placing the cup in the socket.

3. The method of claim 1 wherein the ball and socket joint is a shoulder.

4. The method of claim 1 wherein the three dimensional model is constructed based on non-invasively acquired landmarks.

5. The method of claim 1 wherein the three dimensional model is constructed based on invasively acquired landmarks.

6. The method of claim 1 wherein the three dimensional model is based in part on a neutral positioning of the limb.

7. The method of claim 1 wherein method includes the additional step of determining the stability of the joint.

8. The method of claim 1 wherein method includes the additional step of verifying the three dimensional model.

9. The method of claim 1 wherein the preparing of the limb is conducted by aligning a resection guide relative to a proximal shaft axis, a sagittal plane, and coronal plane as determined by the three dimensional model.

10. The method of claim 9 wherein the aligning of the resection guide is also relative to dimensions of a proposed implant and based on pre-surgically determined changes in geometry of the joint.

11. The method of claim 2 wherein the socket is an acetabulum and wherein the preparing of the socket is conducted by reaming of the acetabulum to a predetermined orientation guided by the surgical navigation system relative to the three dimensional model.

12. The method of claim 2 wherein the socket is an acetabulum and wherein the preparing of the socket is reaming of the acetabulum to a depth guided by the surgical navigation system relative to the three dimensional model.

13. The method of claim 12 wherein the depth relates to a medial wall of the acetabulum.

14. The method of claim 2 wherein the limb is a femur and wherein the preparing of the limb is conducted by broaching the femur to a predetermined depth guided by the surgical navigation system relative to the three dimensional model.

15. The method of claim 2 wherein the limb is a femur and wherein the preparing of the limb is conducted by broaching the femur to a predetermined orientation along a proximal shaft axis, a sagittal plane, and a coronal plane guided by the surgical navigation system relative to the three dimensional model.

16. The method of claim 2 wherein the inserting of the cup is conducted by impacting the cup to a depth guided by the surgical navigation system relative to the three dimensional model.

17. The method of claim 2 wherein the inserting of the cup is conducted by impacting the acetabular cup to an orientation guided by the surgical navigation system using the three dimensional model.

18. The method of claim 2 wherein the socket is an acetabulum and wherein the inserting of the cup is conducted by impacting the cup to a depth that relates to a previously recorded final depth of the prepared acetabulum.

19. The method of claim 2 wherein the socket is an acetabulum and wherein the inserting of the cup is conducted by impacting the cup to an orientation guided by the surgical navigation system using the three dimensional model.

20. The method of claim 18 wherein the inserting of the cup is conducted by impacting the cup to a depth that relates to a previously recorded final depth of the prepared acetabulum.

21. The method of claim 1 wherein the inserting of the stem is conducted by impacting the stem to a depth guided by the surgical navigation system using the three dimensional model.

22. The method of claim 1 wherein the inserting of the stem is conducted by impacting the stem to an orientation along a proximal shaft axis and a sagittal plane and a coronal plane guided by the surgical navigation system using the three dimensional model.

23. The method of claim 1 wherein method includes the additional step of determining the stability of the joint and the joint stability is determined based on the three dimensional model and on a center of the cup and the stem.

24. The method of claim 2 wherein the range of motion is determined based on the three dimensional model and a center of the cup and the stem.

25. The method of claim 1 including the additional step of displaying a result of implant geometry changes on range of motion and joint stability.

26. The method of claim 25 wherein the results of the implant geometry changes on range of motion and stability are determined based on the three dimensional model and a center of the cup and the stem.

27. The method of claim 1 including the additional step of performing a virtual trial using the three dimensional model of the joint and using a database of joint implant components to chose implant components and virtually preparing the joint to receive the implant components.

28. A method of performing a total arthroplasty of a ball and socket joint of a patient using a surgical navigation system wherein the joint has a socket and a limb having a ball shaped head at a proximal end of the limb near the socket comprising the steps of:
constructing a three dimensional model of the joint intra-operatively using the surgical navigation system based on the patient's anatomical landmarks;
preparing the limb to receive a stem using the three dimensional model;
placing the stem in the limb; and
determining the stability of the joint.

29. The method of claim 28 wherein the ball and socket joint is a hip and wherein the method includes the additional steps of:
preparing the socket to receive a cup using the three dimensional model; and placing the cup in the socket.

30. The method of claim 28 wherein the ball and socket joint is a shoulder.

31. The method of claim 28 wherein the three dimensional model is constructed based on non-invasively acquired landmarks.

32. The method of claim 28 wherein the three dimensional model is constructed based on invasively acquired landmarks.

33. The method of claim 28 wherein the three dimensional model is based in part on a neutral positioning of the limb.

34. The method of claim 28 wherein method includes the additional step of determining the range of motion of the joint.

35. The method of claim 28 wherein method includes the additional step of verifying the three dimensional model.

36. The method of claim 28 wherein the preparing of the limb is conducted by aligning a resection guide relative to a proximal shaft axis and a sagittal plane and coronal plane as determined by the three dimensional model.

37. The method of claim 36 wherein the aligning of the resection guide is also relative to dimensions of a proposed implant and based on pre-surgically determined changes in geometry of the joint.

38. The method of claim 29 wherein the socket is an acetabulum and wherein the preparing of the socket is conducted by reaming of the acetabulum to a predetermined orientation guided by the surgical navigation system relative to the three dimensional model.

39. The method of claim 29 wherein the socket is an acetabulum and wherein the preparing of the socket is reaming of the acetabulum to a depth guided by the surgical navigation system relative to the three dimensional model.

40. The method of claim 39 wherein the depth relates to a medial wall of the acetabulum.

41. The method of claim 29 wherein the limb is a femur and wherein the preparing of the limb is conducted by broaching the femur to a predetermined depth guided by the surgical navigation system relative to the three dimensional model.

42. The method of claim 29 wherein the limb is a femur and wherein the preparing of the limb is conducted by broaching the femur to a predetermined orientation along a proximal shaft axis, a sagittal plane, and a coronal plane guided by the surgical navigation system relative to the three dimensional model.

43. The method of claim 29 wherein the inserting of the cup is conducted by impacting the cup to a depth guided by the surgical navigation system relative to the three dimensional model.

43. The method of claim 29 wherein the inserting of the cup is conducted by impacting the acetabular cup to an orientation guided by the surgical navigation system using the three dimensional model.

45. The method of claim 29 wherein the socket is an acetabulum and wherein the inserting of the cup is conducted by impacting the cup to a depth that relates to a previously recorded final depth of the prepared acetabulum.

46. The method of claim 29 wherein the socket is an acetabulum and wherein the inserting of the cup is conducted by impacting the cup to an orientation guided by the surgical navigation system using the three dimensional model.

47. The method of claim 45 wherein the inserting of the cup is conducted by impacting the cup to a depth that relates to a previously recorded final depth of the prepared acetabulum.

48. The method of claim 28 wherein the inserting of the stem is conducted by impacting the stem to a depth guided by the surgical navigation system using the three dimensional model.

49. The method of claim 28 wherein the inserting of the stem is conducted by impacting the stem to an orientation along a proximal shaft axis, a sagittal plane, and a coronal plane guided by the surgical navigation system using the three dimensional model.

50. The method of claim 29 wherein method includes the additional step of determining the range of motion of the joint and the range of motion is determined based on the three dimensional model and on a center of the cup and the stem.

51. The method of claim 28 including the additional step of displaying a result of implant geometry changes on joint stability.

52. The method of claim 51 wherein the results of the implant geometry changes on stability are determined based on the three dimensional model and a center of the cup and the stem.

53. The method of claim 28 including the additional step of performing a virtual trial using the three dimensional model of the joint and using a database of joint implant components to chose implant components and virtually preparing the joint to receive the implant components.

54. A system for assisting in the performance of total arthroplasty of a ball and socket joint on a patient comprising:
a surgical navigation system;
a first circuit to construct a three dimensional model of the ball and socket joint intra-operatively using the surgical navigation system based on the patient's anatomical landmarks;
a first tool to prepare a limb to receive a stem, wherein the first tool can be tracked by the surgical navigation system to determine the position and orientation of the first tool and wherein the position and orientation of the first tool is tracked relative to the three dimensional model;
a second tool to place the stem in the limb, wherein the second tool can be tracked by the surgical navigation system to determine the position and orientation of the second tool and wherein the position and orientation of the second tool is tracked relative to the three dimensional model; and
a second circuit to determine a joint range of motion.

55. The system of claim 54 wherein the ball and socket joint is a hip joint and wherein the limb is a femur, including a third tool to prepare an acetabulum that can be tracked by the surgical navigation system to determine the position and orientation of the third tool relative to the three dimensional model; and a fourth tool to place an implant into the prepared acetabulum wherein the fourth tool can be tracked by the surgical navigation system to determine the position and orientation of the fourth tool relative to the three dimensional model.

56. The system of claim 54 wherein the first circuit constructs the three dimensional model based on non-invasively acquired landmarks.

57. The system of claim 54 wherein the first circuit constructs the three dimensional model based on invasively acquired landmarks.

58. The system of claim 55 including a resection guide to assist in the resection of the femur wherein the resection guide can be tracked by the surgical navigation system to align the resection guide relative to a proximal shaft axis, a sagittal plane, and a coronal plane determined by the three dimensional model.

59. The system of claim 55 including a resection guide to assist in the resection of a neck of the femur that can be tracked by the surgical navigation system relative to a proximal shaft axis, a sagittal plane, and coronal plane and relative to the dimensions of a proposed implant and on pre-surgically determined changes in geometry of the hip joint.

60. The system of claim 55 wherein the third tool reams the acetabulum to a depth guided by the surgical navigation system relative to the three dimensional model.

61. The system of claim 55 wherein the third tool reams the acetabulum to a predetermined orientation guided by the surgical navigation system relative to the three dimensional model.

62. The system of claim 55 wherein the first tool broaches the femur to a predetermined depth guided by the surgical navigation system relative to the three dimensional model.

63. The system of claim 55 wherein the first tool broaches the femur to a predetermined orientation along a proximal shaft axis, a sagittal plane, and a coronal plane guided by the surgical navigation system relative to the three dimensional model.

64. The system of claim 55 wherein the fourth tool inserts the implant to a depth guided by the surgical navigation system relative to the three dimensional model.

65. The system of claim 55 wherein the fourth tool inserts the implant to an orientation guided by the surgical navigation system using the three dimensional model.

66. The system of claim 54 wherein the second tool inserts the stem to a depth guided by the surgical navigation system using the three dimensional model.

67. The system of claim 54 wherein the second tool inserts the stem to an orientation along a proximal shaft axis, a sagittal plane, and a coronal plane guided by the surgical navigation system using the three dimensional model.

68. The system of claim 55 wherein the second circuit determines hip joint range of motion based on the three dimensional model and a center of the implant and the stem.

69. The system of claim 55 wherein a third circuit determines the stability of the hip joint based on the three dimensional model and a center of the implant and the stem.

70. The system of claim 69 including a fourth circuit to display a result of implant geometry changes on range of motion and hip stability.

71. The system of claim 54 that includes a third circuit to verify the three dimensional model.

72. A system for assisting in the performance of total arthroplasty of a ball and socket joint on a patient comprising:
a surgical navigation system;
a first circuit to construct a three dimensional model of the ball and socket joint intra-operatively using the surgical navigation system based on the patient's anatomical landmarks;
a first tool to prepare a limb to receive a stem, wherein the first tool can be tracked by the surgical navigation system to determine the position and orientation of the first tool and wherein the position and orientation of the first tool is tracked relative to the three dimensional model;
a second tool to place the stem in the limb, wherein the second tool can be tracked by the surgical navigation system to determine the position and orientation of the second tool and wherein the position and orientation of the second tool is tracked relative to the three dimensional model; and
a second circuit to determine a stability of the joint.

73. The system of claim 72 wherein the ball and socket joint is a hip joint and wherein the limb is a femur, including a third tool to prepare an acetabulum that can be tracked by the surgical navigation system to determine the position and orientation of the third tool relative to the three dimensional model; and a fourth tool to place an implant into the prepared acetabulum wherein the fourth tool can be tracked by the surgical navigation system to determine the position and orientation of the fourth tool relative to the three dimensional model.

74. The system of claim 72 wherein the first circuit constructs the three dimensional model based on non-invasively acquired landmarks.

75. The system of claim 72 wherein the first circuit constructs the three dimensional model based on invasively acquired landmarks.

76. The system of claim 73 including a resection guide to assist in the resection of the femur wherein the resection guide can be tracked by the surgical navigation system to align the resection guide relative to a proximal shift axis, a sagittal plane, and a coronal plane determined by the three dimensional model.

77. The system of claim 73 including a resection guide to assist in the resection of a neck of the femur that can be tracked by the surgical navigation system relative to a proximal shaft axis, a sagittal plane, and coronal plane and relative to the dimensions of a proposed implant and on pre-surgically determined changes in geometry of the hip joint.

78. The system of claim 73 wherein the third tool reams the acetabulum to a depth guided by the surgical navigation system relative to the three dimensional model.

79. The system of claim 73 wherein the third tool reams the acetabulum to a predetermined orientation guided by the surgical navigation system relative to the three dimensional model.

80. The system of claim 73 wherein the fist tool broaches the femur to a predetermined depth guided by the surgical navigation system relative to the three dimensional model.

81. The system of claim 73 wherein the first tool broaches the femur to a predetermined orientation along a proximal shaft axis, a sagittal plane, and a coronal plane guided by the surgical navigation system relative to the three dimensional model.

82. The system of claim 73 wherein the fourth tool inserts the implant to a depth guided by the surgical navigation system relative to the three dimensional model.

83. The system of claim 73 wherein the fourth tool inserts the implant to an orientation guided by the surgical navigation system using the three dimensional model

84. The system of claim 72 wherein the second tool inserts the stem to a depth guided by the surgical navigation system using the three dimensional model.

85. The system of claim 72 wherein the second tool inserts the stem to an orientation along a proximal shaft axis, a sagittal plane, and a coronal plane guided by the surgical navigation system using the three dimensional model.

86. The system of claim 73 wherein the second circuit determines the stability of the hip joint based on the three dimensional model and a center of the implant and the stem.

87. The system of claim 73 including a fourth circuit to display a result of implant geometry changes on range of motion and hip stability.

88. The system of claim 72 that includes a third circuit to verify the three dimensional model.

89. A method of performing a total arthroplasty of a ball and socket joint of a patient using a surgical navigation system wherein the joint has a socket and a limb having a ball shaped head at a proximal end of the limb near the socket comprising the steps of:
constructing a three dimensional model of the joint;
providing a virtual trial of the joint using the three dimensional model of the joint and data relating to implant components chosen from a database of joint implant components;
preparing a limb to receive a stem implant using the three dimensional model; and
placing the stem implant within the prepared limb.

90. The method of claim 89 wherein the ball and socket joint is a hip and wherein the method includes the additional steps of:
preparing the socket to receive a cup using the three dimensional model; and
placing the cup in the socket.

91. The method of claim 89 wherein the ball and socket joint is a shoulder.

92. The method of claim 89 wherein the three dimensional model is constructed based on non-invasively acquired landmarks.

93. The method of claim 89 wherein the three dimensional model is constructed based on invasively acquired landmarks.

94. The method of claim 89 wherein the three dimensional model is based in part on a neutral positioning of the limb.

95. The method of claim 89 wherein method includes the additional step of determining the stability of the joint.

96. The method of claim 89 wherein method includes the additional step of verifying the three dimensional model.

97. The method of claim 89 wherein the three dimensional model of the joint is constructed intra-operatively using the surgical navigation system based on landmarks on a patient.

98. The method of claim 89 wherein the three dimensional model of the joint is constructed based on pre-operative scan data.

99. The method of claim 89 wherein the virtual trial is conducted prior to the preparation of the joint.

100. The method of claim 89 wherein the virtual trial is conducted at any time during the preparation of the joint.

101. The method of claim 89 wherein the virtual trial is conducted after to the preparation of the joint.

102. The method of claim 89 wherein a trial reduction is performed prior to placing an implant into the prepared joint.

103. The method of claim 89 including the additional step of displaying a result of implant geometry changes on joint stability.

104. The method of claim 103 wherein the range of motion is determined based on the three dimensional model and a center of the stem.

105. The method of claim 89 including the additional step of displaying a result of implant geometry changes on joint range of motion.

106. The method of claim 105 wherein the results of implant geometry changes on range of motion are determined based on the three dimensional model and a center of the stem.

107. A system for performing a total arthroplasty of a ball and socket joint of a patient comprising:
a surgical navigation system;
a first circuit to construct a three dimensional model of the joint;
a second circuit to provide a virtual trial of the joint using the three dimensional model of the joint and data relating to implant components chosen from a database of joint implant components;
a first tool to prepare a limb to receive a stem, wherein the first tool can be tracked by the surgical navigation system to determine the position and orientation of the first tool and wherein the position and orientation of the first tool is tracked relative to the three dimensional model; and
a second tool to place the stem in the limb, wherein the second tool can be tracked by the surgical navigation system to determine the position and orientation of the second tool and wherein the position and orientation of the second tool is tracked relative to the three dimensional model.

108. The system of claim 107 wherein the ball and socket joint is a hip joint and wherein the limb is a femur, including a third tool to prepare an acetabulum that can be tracked by the surgical navigation system to determine the position and orientation of the third tool relative to the three dimensional model; and a fourth tool to place an implant into the prepared acetabulum wherein the fourth tool can be tracked by the surgical navigation system to determine the position and orientation of the fourth tool relative to the three dimensional model.

109. The system of claim 107 wherein the ball and socket joint is a shoulder.

110. The system of claim 107 wherein the three dimensional model is constructed based on non-invasively acquired landmarks.

111. The system of claim 107 wherein the three dimensional model is constructed based on invasively acquired landmarks.

112. The system of claim 107 wherein the three dimensional model is based in part on a neutral positioning of the limb.

113. The system of claim 107 including a third circuit to determine the stability of the joint.

114. The system of claim 107 including a third circuit to verifying the three dimensional model.

115. The system of claim 107 wherein the three dimensional model of the joint is constructed intra-operatively using the surgical navigation system based on landmarks on a patient.

116. The system of claim 107 wherein the three dimensional model of the joint is constructed based on pre-operative scan data.

117. The system of claim 107 including a third circuit to display a result of implant geometry changes on joint stability.

118. The system of claim 117 wherein the joint stability is determined based on the three dimensional model and a center of the stem.

119. The system of claim 107 including a third circuit to display a result of implant geometry changes on joint range of motion.

120. The system of claim 119 wherein the results of implant geometry changes on range of motion are determined based on the three dimensional model and a center of the stem.

121. A device to be used with a tracking device to locate the center line of a canal of a limb comprising of an elongate body that can be inserted into the canal, a series of outwardly biased surfaces spaced around the elongate body and an interface attached to the body to enable a locating device capable of being tracked by a surgical navigation system to be affixed to the device.

122. A device to be used with a tracking device to locate a level of resection of a neck of a limb comprising a flat guide surface, a handle, and an interface attached to the handle to enable a locating device capable of being tracked by a surgical navigation system to be attached to the device.
